(19) United States
(12) Patent Application Publication
(10) Pub. No.: US 2025/0040881 A1
(43) Pub. Date: Feb. 6, 2025

(54) TREATMENT SUPPORT DEVICE

(71) Applicant: SHIMADZU CORPORATION, Kyoto-shi (JP)

(72) Inventors: Akihiro ISHIKAWA, Kyoto-shi (JP); Toru YAMAGUCHI, Kyoto-shi (JP)

(21) Appl. No.: 18/718,812

(22) PCT Filed: Dec. 13, 2021
(86) PCT No.: PCT/JP2021/045838

§ 371 Date: Jun. 12, 2024

(51) Int. Cl. A61B 5/00

(52) U.S. Cl. CPC A61B 5/4848; A61B 5/0071

(57) ABSTRACT

This treatment support device is equipped with a fluorescence detection unit configured to detect fluorescence emitted from a fluorescent substance of a drug excited by therapeutic light used in photoimmunotherapy, a change information generation unit configured to selectively acquire first signal information corresponding to light in a first wavelength band, which is a wavelength band including a wavelength of approximately 770 nm, among signal waveforms of the fluorescence detected by the fluorescence detection unit, and generate first fluorescence change information, which is information on a change in fluorescence intensity in the first wavelength band, based on the acquired first signal information, and a notification unit (display unit) that notifies the treatment progress based on the first fluorescence change information.

### TECHNICAL FIELD

The present invention relates to a treatment support device.

### BACKGROUND ART

Conventionally, a treatment support device is known in which the support of a treatment and the treatment by photoimmunotherapy for killing cancer cells are performed by irradiating a drug containing a fluorescent substance that has been administered to a subject's body with therapeutic light in a specific wavelength band. Such a treatment support device is disclosed in, for example, Japanese Unexamined Patent Application Publication No. WO 2021/038726.

The above-described International Publication No. WO 2021/038726 discloses a treatment support device equipped with a light source that emits therapeutic light toward a treatment site of a subject (patient) to which a drug containing a fluorescent substance has been administered and a fluorescence detection unit that detects the intensity of the fluorescence (fluorescence intensity), the fluorescence being generated from the fluorescent substance in the drug. Note that when continuously being irradiated with therapeutic light, a drug undergoes a photochemical reaction, causing injury to cancer cells, which results in the ceased emission of fluorescence. Therefore, although not specifically described in the above-described International Publication No. WO 2021/038726, in such a conventional treatment support device, a user, such as a doctor, confirms the progress of the treatment by the attenuation of fluorescence intensity in accordance with the irradiation time of the therapeutic light.

### PRIOR ART DOCUMENT

### Patent Document

Patent Document 1: International Publication No. WO 2021/038726

### SUMMARY OF THE INVENTION

### Problems to be Solved by the Invention

Here, the attenuation of fluorescence intensity, which is used by a user, such as a doctor, to confirm the progress of treatment, is based on the change in the intensity of fluorescence (fluorescence intensity) detected by the fluorescence detection unit due to the increase in the treatment time and is based on the change in the signal waveform of the fluorescence detected by the fluorescence detection unit due to the increase in the treatment time. Therefore, there is a need for a treatment support device that can acquire the change in the fluorescence signal waveform due to the increase in the treatment time with high sensitivity, thereby enabling the sensitive acquisition of the progress of treatment by photoimmunotherapy.

The present invention has been made to solve the above-described problems. One object of the present invention is to provide a treatment support device capable of sensitively acquiring changes in a fluorescence signal waveform due to an increase in treatment time, thereby sensitively acquiring the progress of treatment by photoimmunotherapy.

### Means for Solving the Problems

A treatment support device according to one aspect of the present invention comprises:
a fluorescence detection unit configured to detect, in a photoimmunotherapy for killing cancer cells by irradiating a drug containing a fluorescent substance that has been administered to a body of a subject with therapeutic light in a specific wavelength band, fluorescence emitted from the fluorescent substance of the drug excited by the therapeutic light;
a change information generation unit configured to selectively acquire first signal information corresponding to light in a first wavelength band that is a wavelength band including a wavelength of 770 nm or in the vicinity thereof, among signal waveforms of fluorescence emitted from the fluorescent substance of the drug detected by the fluorescence detection unit, and generate first fluorescence change information that is information on a change in fluorescence intensity in the first wavelength band, based on acquired first signal information; and
a notification unit configured to notify progress of a treatment based on irradiation of the therapeutic light, based on the first fluorescence change information

### Effects of the Invention

Here, the present inventor focused on the change in the signal waveform of the fluorescence emitted from the fluorescent substance of the drug excited by the therapeutic light in photoimmunotherapy, the change occurring due to an increase in treatment time. After careful consideration, the present inventor found that the change in the fluorescence signal waveform due to the increase in treatment time in the wavelength band at or around 770 nm is larger than the change in the fluorescence signal waveform due to the increase in treatment time in other wavelength bands, and thus conceived the present invention.

In the treatment support device according to a first aspect of the present invention, as described above, the change information generation unit selectively acquires first signal information corresponding to the light in the first wavelength band, which is a wavelength band including a wavelength in the vicinity of 770 nm. The change information generation unit generates first fluorescence change information, which is information on the change in fluorescence intensity in the first wavelength band, based on the acquired first signal information. With this, the information (first fluorescence change information) on the change in fluorescence intensity in the wavelength band in the vicinity of 770 nm, where the change in the signal waveform of fluorescence due to the increase in treatment time is found to be larger than that in other wavelength bands, is selectively acquired. This excludes the information on the change in fluorescence intensity in the signal waveform of the fluorescence emitted from the fluorescent substance of the drug, where the change in the signal waveform of the fluorescence due to the increase in treatment time is small. As a result, compared with the case of acquiring the information on the change in the fluorescence intensity of the entire waveform of the fluorescence emitted from the fluorescent substance of the drug, it is possible to acquire the change in the signal waveform of the fluorescence due to the increase in treatment time with high sensitivity. With this, it is possible to acquire the change in the signal waveform of the fluorescence due to the increase in treatment time with high sensitivity, thereby acquiring the treatment progress by photoimmunotherapy with high sensitivity. Furthermore, based on the first fluorescence change information sensitively reflecting the change in the fluorescence signal waveform due to the increase in treatment time, the progress of the treatment is notified by the notification unit. With this, through the notification by the notification unit, the user, such as a doctor, can grasp the progress of the treatment acquired with high sensitivity.

### BRIEF DESCRIPTION OF THE DRAWINGS

FIG. 1 is a diagram for explaining the mechanism of action of a drug in photoimmunotherapy.

FIG. 2 is a diagram for explaining the mechanism of action of a drug in photoimmunotherapy.

FIG. 3 is a schematic diagram showing the overall configuration of a treatment support device according to one embodiment of the present invention.

FIG. 4 is a diagram showing one example of a fluorescence distribution image.

FIG. 5 is a diagram showing one example of a visible light image.

FIG. 6 shows one example of a composite image.

FIG. 7 shows one example of the attenuation of fluorescence intensity due to an increase in treatment time.

FIG. 8 is a waveform diagram showing signal waveforms of fluorescence at each of the treatment times t1 to t6.

FIG. 9 is a waveform diagram showing waveforms after normalizing each of the fluorescence signal waveforms shown in FIG. 8.

FIG. 10 is an enlarged waveform diagram showing the second wavelength band and the vicinity thereof shown in FIG. 9.

FIG. 11 is a diagram that compares the relative changes in the fluorescence intensity based on the light in the first wavelength band and the fluorescence intensity based on the light in wavelength bands other than the first wavelength band, with respect to each treatment time.

FIG. 12 is a diagram showing one example of the first change information.

FIG. 13 is a diagram showing one example of the second change information.

FIG. 14 is a diagram showing one example of a treatment progress index.

FIG. 15 is a diagram showing one example of a display by the display unit of a treatment support device according to one embodiment of the present invention.

FIG. 16 is a diagram showing another example of display by the display unit of the treatment support device according to one embodiment of the present invention.

FIG. 17 is a schematic diagram showing a first modification of the treatment support device according to one embodiment of the present invention.

FIG. 18 is a schematic diagram showing a second modification of the treatment support device according to one embodiment of the present invention.

FIG. 19 is a schematic diagram showing a third modification of the treatment support device according to one embodiment of the present invention.

### EMBODIMENTS FOR CARRYING OUT THE INVENTION

Hereinafter, some embodiments in which the present invention is embodied will be described based on the attached drawings.

### (Photoimmunotherapy)

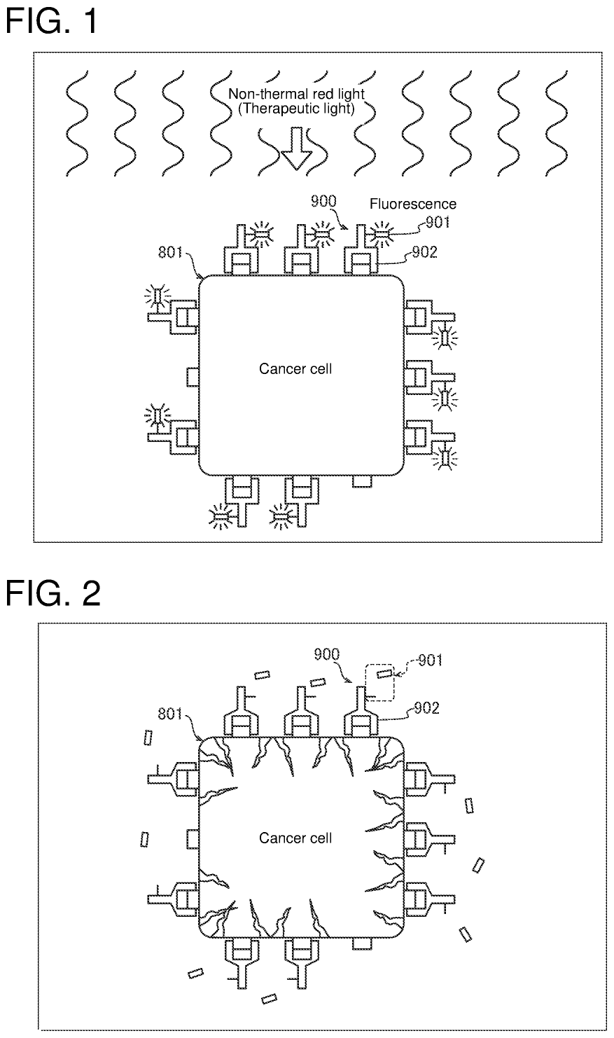

First, photoimmunotherapy (PIT: Photoimmunotherapy) will be explained with reference to FIG. 1 and FIG. 2. In photoimmunotherapy, as shown in FIG. 1, a treatment to kill cancer cells 801 is performed using a drug 900 that selectively binds to cancer cells 801. The drug 900 includes a fluorescent substance 901 that emits fluorescence and an antibody 902. The fluorescent substance 901 of the drug 900 is a substance that is excited and emits fluorescence when irradiated with light in a specific wavelength band, and a substance that undergoes a photochemical reaction when continuously irradiated with light in the specific wavelength band. The fluorescent substance 901 is, for example, a chemical substance such as IRDye700 (registered trademark).

In photoimmunotherapy, a treatment to kill cancer cells 801 is performed based on continuous irradiation of a drug 900 containing a fluorescent substance 901 with therapeutic light in a specific wavelength band. Specifically, by continuously irradiating the drug 900, which contains the fluorescent substance 901, with therapeutic light in a specific wavelength band, the fluorescent substance 901 of the drug 900 emits fluorescence and undergoes a photochemical reaction to change the chemical structure of the fluorescent substance 901 (see FIG. 2). This change in the chemical structure of the fluorescent substance 901 causes a change in the steric structure of the antibody 902. The change in the steric structure of the antibody 902 bound to the cancer cell 801 causes damage to the cell membrane of the bound cancer cell 801, thereby destroying (killing) the cancer cell 801.

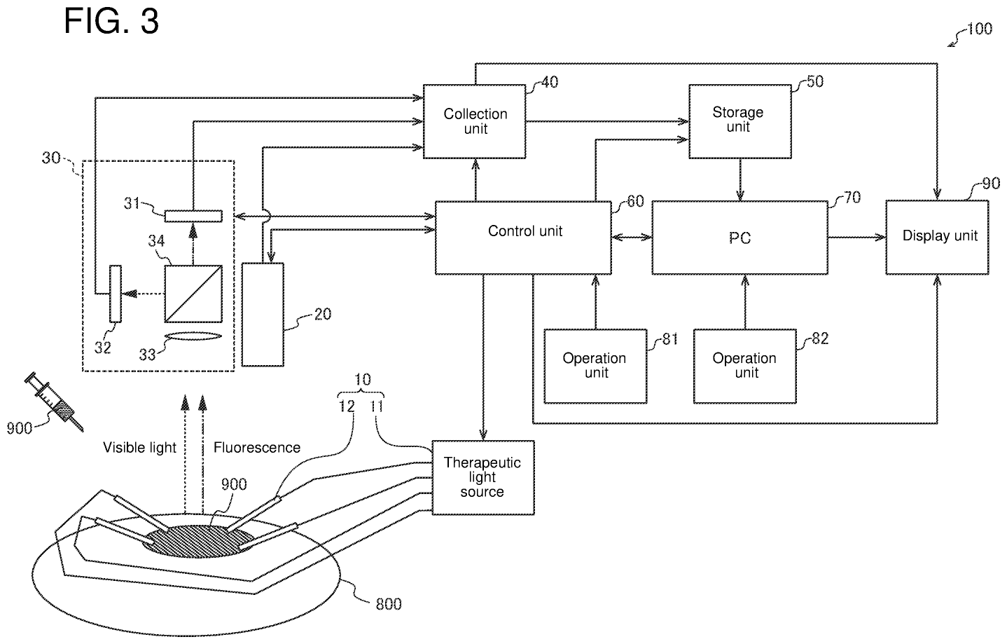

Note that in the treatment by photoimmunotherapy, therapeutic light (excitation light) corresponding to the type of the fluorescent substance 901 of the drug 900 that has been administered to the patient 800 (see FIG. 3) is irradiated. The therapeutic light (excitation light) irradiated on the drug 900 in a treatment by photoimmunotherapy is light in a wavelength band in which a fluorescent substance 901 of the drug 900 used in the treatment undergoes a photochemical reaction in a wavelength region of 600 nm or more and 2,500 nm or less, which is a region from a part of visible light to near-infrared light. In this embodiment, IRDye700 (registered trademark) is used as the fluorescent substance 901 of the drug 900, and in the treatment by photoimmunotherapy, the drug 900 (fluorescent substance 901) is irradiated with light in which the peak position of the wavelength is 600 nm or more and 700 nm or less. Specifically, in the treatment by photoimmunotherapy, non-thermal red light (near-infrared light) with a wavelength peak position of approximately 690 nm is irradiated onto the drug 900 (fluorescent substance 901).

Thus, in photoimmunotherapy, a treatment to kill cancer cells 801 is performed based on irradiating therapeutic light in a specific wavelength band onto the drug 900 containing a fluorescent substance 901 that has been administered to the body of the patient 800.

### (Treatment Support Device)

The treatment support device 100 (see FIG. 3) according to this embodiment is a device that provides treatment support for photoimmunotherapy. Specifically, the treatment support device 100 is configured to emit therapeutic light (excitation light) to the patient 800 and detect fluorescence emitted from the fluorescent substance 901 of the drug 900 that has been administered to the patient 800. The treatment support device 100 is configured such that in addition to support treatment by photoimmunotherapy, it is possible to necrotize (perform treatment by photoimmunotherapy) cancer cells 801 by continuously emitting therapeutic light, which is light in a specific wavelength band according to the fluorescent substance 901 of the drug 900. In other words, the treatment support device 100 of this embodiment also serves as a treatment device that performs treatment by photoimmunotherapy. Note that the patient 800 is one example of the “subject” recited in claims. Further, the patient 800 may be an animal other than a human.

### (Configuration for Irradiation of Therapeutic Light)

The treatment support device 100 is equipped with an irradiation unit 10, as shown in FIG. 3. The irradiation unit 10 is configured to emit therapeutic light (excitation light), which is light in a specific wavelength band that excites the fluorescent substance 901 of the drug 900 administered to the patient 800.

In the treatment by photoimmunotherapy, the irradiation unit 10 is configured to irradiate the drug 900, which contains the fluorescent substance 901 administered to the body of the patient 800, with the therapeutic light (excitation light). The irradiation unit 10 is configured to emit light in a wavelength band including a wavelength of 690 nm as therapeutic light. Further, as shown in FIG. 3, the irradiation unit 10 includes a therapeutic light source 11 and a plurality of therapeutic probes 12.

The therapeutic light source 11 is configured to emit therapeutic light (excitation light), which is light in a specific wavelength band that excites the fluorescent substance 901 contained in the drug 900. The therapeutic light source 11 includes a semiconductor laser (LD: Laser Diode) or a light emitting diode (LED: Light Emitting Diode).

The therapeutic probe 12 is configured to be inserted into the body of a patient 800 and to emit therapeutic light in the body of the patient 800. The therapeutic probe 12 includes an optical fiber that guides the light emitted from the therapeutic light source 11. The therapeutic probe 12 is inserted along a cylindrical guide (not shown), such as a diffuser, which is formed of a transparent member such as a glass member inserted into the body of the patient 800, toward a position (treatment site) that is a treatment portion in the body of the patient 800.

The user, such as a doctor, should know in advance the location (affected part) of the cancer by an MRI (Magnetic Resonance Image), an X-ray CT (Computed Tomography), or an ultrasound echo. The user, such as a doctor, inserts the therapeutic probe 12 into the body of the patient 800 while confirming the position of the cancer by an ultrasound echo or other means. The therapeutic probe 12 is configured to guide and emit the therapeutic light from the therapeutic light source 11 within the body of the patient 800. At the time of the treatment by photoimmunotherapy, therapeutic light corresponding to the type of the fluorescent substance 901 of the drug 900 administered to the patient 800 is emitted to the treatment site (cancer cells 801) of the patient 800 by the irradiation unit 10. With this, the fluorescent substance 901 of the drug 900 is excited by the therapeutic light.

As described above, the treatment support device 100 according to this embodiment enables the treatment (treatment by photoimmunotherapy) of cancer cells 801 by continuously emitting therapeutic light (excitation light), which is light in a specific wavelength band that excites the fluorescent substance 901 of the drug 900, in the body of the patient 800 by the irradiation unit 10 (therapeutic light source 11 and therapeutic probe 12).

### (Configuration for Detection of Fluorescence and Visible Light)

The treatment support device 100 is equipped with a fluorescence detection unit 20, an imaging unit 30, a collection unit 40, and a storage unit 50, as shown in FIG. 3.

The fluorescence detection unit 20 is configured to detect the fluorescence emitted from the fluorescent substance 901 of the drug 900 excited by the therapeutic light emitted from the irradiation unit 10 in photoimmunotherapy. Further, the fluorescence detection unit 20 includes a spectrometer, such as a spectrum meter, and is configured to detect the fluorescence emitted from the fluorescent substance 901 of the drug 900 by sequential spectroscoping for each predetermined wavelength band. Further, the detection of the fluorescence emitted from the fluorescent substance 901 of the drug 900 by the fluorescence detection unit 20 is configured to be synchronized with the irradiation (irradiation timing) of the therapeutic light by the irradiation unit 10. The details of the detection of the fluorescence by the fluorescence detection unit 20 will be described later.

The imaging unit 30 is equipped with a fluorescence imaging unit 31 and a visible light imaging unit 32. Further, the imaging unit 30 is equipped with a lens 33 and a prism 34. As shown in FIG. 3, the fluorescence imaging unit 31 is provided separately from the fluorescence detection unit 20.

The fluorescence imaging unit 31 is configured to image the distribution of the fluorescence emitted from the fluorescent substance 901 of the drug 900 excited by the therapeutic light. The fluorescence imaging unit 31 is configured to detect the fluorescence emitted from the fluorescent substance 901 of the drug 900 when the therapeutic light is irradiated. The fluorescence imaging unit 31 images the fluorescence emitted from the fluorescent substance 901 at a predetermined frame rate, such as the NTSC (National Television System Committee) standard frame rate. The fluorescence imaging unit 31 includes an image sensor such as a CMOS (Complementary Metal Oxide Semiconductor) image sensor and a CCD (Charge Coupled Device) image sensor.

The visible light imaging unit 32 is configured to detect visible light including the reflected light from the patient 800. The visible light imaging unit 32 includes an image sensor such as a CMOS image sensor and a CCD image sensor. The visible light imaging unit 32 images the visible light (reflected light) reflected from the patient 800 at a predetermined frame rate, such as the NTSC standard frame rate. In this embodiment in which IRDye700 (registered trademark) is used as the fluorescent substance 901 of the drug 900, the visible light imaging unit 32 detects the visible light including the therapeutic light (excitation light) irradiated from the irradiation unit 10.

The lens 33 is configured so that the fluorescence emitted from the fluorescent substance 901 of the drug 900 and the visible light (reflected light) including the therapeutic light irradiated from the irradiation unit 10 are incident on the lens. The visible light, including the fluorescence and the therapeutic light, incident on the lens 33 is converged by the lens 33 and incident on the prism 34.

The prism 34 is configured to separate the incoming light, and the visible light, including the fluorescence and the therapeutic light, incident on the lens 33 is separated by the prism 34. The fluorescence separated by the prism 34 is configured to form an image at the fluorescence imaging unit 31. Further, the visible light including the therapeutic light separated by the prism 34 is configured to form an image at the visible light imaging unit 32.

The fluorescence imaging unit 31 is configured to detect the light (fluorescence) with a wavelength of 700 nm or more by the wavelength selectivity of the optical filter. The IRDye700 (registered trademark), which is the fluorescent substance 901 of the drug 900, is excited by light with wavelengths of 600 nm or more and 700 nm or less, and emits light with peaks at wavelengths of around 730 nm and around 770 nm, as fluorescence, as will be described below. In other words, the fluorescence imaging unit 31 is configured to selectively detect the light including the wavelength band of the fluorescence emitted from the fluorescent substance 901 in the drug 900.

The visible light imaging unit 32 is configured to detect visible light including the therapeutic light, based on light of a wavelength of 400 nm or more and 700 nm or less, including the wavelength band of the therapeutic light (excitation light) and the wavelength band of the visible light, by the wavelength selectivity of the optical filter. In this embodiment, non-thermal red light in a wavelength band of 600 nm and more and 700 nm and less, and with a peak position of about 690 nm, is emitted as the therapeutic light (excitation light). Thus, the visible light imaging unit 32 is configured to selectively detect light that includes the wavelength band of the therapeutic light irradiated by the irradiation unit 10 (therapeutic probe 12).

The collection unit 40 includes a processor such as a GPU (Graphics Processing Unit) or an FPGA (Field-Programmable Gate Array) configured for image processing.

Each of the fluorescence signal detected by the fluorescence detection unit 20, the fluorescence signal detected by the fluorescence imaging unit 31, and the signal based on the visible light detected by the visible light imaging unit 32 are input to the collection unit 40 as electrical signals. Further, the collection unit 40 is configured to perform collection or collection stoppage of fluorescence signals and also perform collection or collection stoppage of signals based on the visible light, under the control of the control unit 60.

The storage unit 50 includes, for example, a non-volatile memory, a hard disk drive (HDD: Hard Disk Drive), or an SSD (Solid State Drive). As a result, the storage unit 50 is configured to enable long-term storage (retention) of data for each of the fluorescence signal detected by the fluorescence detection unit 20, the fluorescence signal detected by the fluorescence imaging unit 31, and the signal based on visible light detected by the visible light imaging unit 32. Note that the storage unit 50 may include a database on a network connected to a network outside the treatment support device 100.

### (Configuration for Control of Treatment Support Device)

Further, as shown in FIG. 3, the treatment support device 100 is equipped with a control unit 60, a PC (Personal Computer) 70, an operation unit 81, an operation unit 82, and a display unit 90. Note that the PC 70 may be integrally configured with the collection unit 40.

The control unit 60 includes a control board (circuit board) on which a CPU (Central Processing Unit), a ROM (Read Only Memory), a RAM (Random Access Memory), and other components are mounted. The control unit 60 is configured to control the entire treatment support device 100. Note that the control unit 60 and the PC 70 may be configured integrally.

The control unit 60 is configured to perform the control of the irradiation of the therapeutic light by the irradiation unit 10. The control unit 60 is configured to perform the control of turning on and off the therapeutic light source 11 (starting and stopping the irradiation of the therapeutic light). Further, the control unit 60 is configured so that a user, such as a doctor, can perform controls such as starting and stopping the irradiation of the therapeutic light (switching the irradiation of the therapeutic light on and off) by operating the operation unit 81 or the operation unit 82, as will be described later.

The PC 70 is a computer that includes a CPU, a ROM, and a RAM. Note that the PC 70 is one example of the “change information generation unit” as recited in claims. The PC 70 is configured to perform the analysis of the fluorescence signal (signal waveform) detected by the fluorescence detection unit 20. The PC 70 is configured to acquire first fluorescence change information C1, second fluorescence change information C2, and a treatment progress index C3, as will be described later, based on the fluorescence detection results (signal waveforms) by the fluorescence detection unit 20. Further, the PC 70 is configured to perform an analysis of the fluorescence signal (image data based on fluorescence) detected by the fluorescence imaging unit 31 and analysis of the signal (image data based on the visible light) based on the visible light, including therapeutic light, detected by the visible light imaging unit 32.

The operation units 81 and 82 are user interfaces for operating the treatment support device 100. The operation units 81 and 82 include, for example, a remote control, a touch panel, a keyboard, or a mouse. Note that a touch panel serving as the operation unit 81 or 82 may be provided on the display unit 90. In other words, the operation unit 81 or 82 and the display unit 90 may be integrally configured. Note that the PC 70 may be integrally configured with the display unit 90. Further, the operation unit 81 and the operation unit 82 may be integrally configured.

The operation unit 81 is configured to accept operations related to the control of the treatment support device 100 by the control unit 60. The operations related to the control of the treatment support device 100 by the control unit 60 include, for example, operations to start and stop (switching on and off the irradiation of the therapeutic light) the irradiation of the therapeutic light, operations to start and stop the detection by the fluorescence detection unit 20, and operations to start and stop the detection by the imaging unit 30 (fluorescence imaging unit 31 and visible light imaging unit 32).

Further, the operation unit 82 is configured to accept, such as, e.g., operations related to analyses by the PC, the analysis of the signal waveform detected by the fluorescence detection unit 20, the analysis of the fluorescence signal (image data based on fluorescence), and the analysis of the signal (image data based on the visible light) based on the visible light, including the therapeutic light. Further, the operation unit 82 is configured to accept operations related to switching the display of the display unit 90.

The display unit 90 is configured by, for example, a liquid crystal display or an organic EL display. The display unit 90 is connected to the control unit 60 and the PC 70 by a video interface such as, e.g., an HDMI (registered trademark). Note that the display unit 90 is one example of the “notification unit” recited in claims.

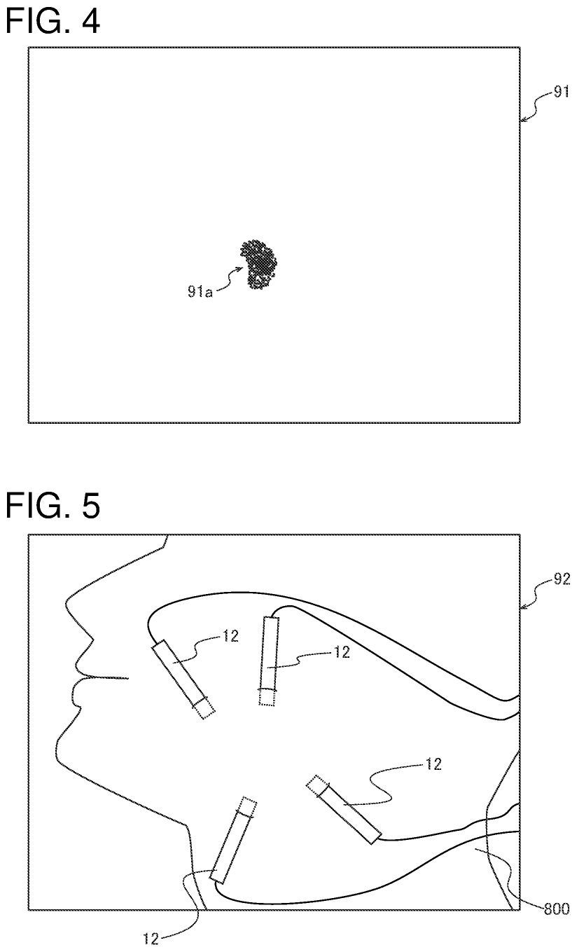

The display unit 90 is configured to display a fluorescence distribution image 91 (see FIG. 4). The fluorescence distribution image 91 is an image showing the distribution state of the fluorescence emitted from the fluorescent substance 901 of the drug 900. The fluorescence distribution image 91 is generated based on the fluorescence signal (image data based on fluorescence) detected by the fluorescence imaging unit 31. A user, such as a doctor, can confirm the accumulation of the drug 900 containing the fluorescent substance 901 bound to the cancer cells 801 by the fluorescence distribution 91a in the fluorescence distribution image 91.

Further, the display unit 90 is configured to display a visible light image 92 (see FIG. 5). The visible light image 92 is an image based on visible light that includes therapeutic light. The visible light image 92 is generated based on the signal (visible light-based image data) based on the visible light detected by the visible light imaging unit 32. The user, such as a doctor, can confirm the position of the therapeutic probe 12 inserted into the cancer patient 800 and the therapeutic light emitted from the therapeutic probe 12 by the visible light image 92.

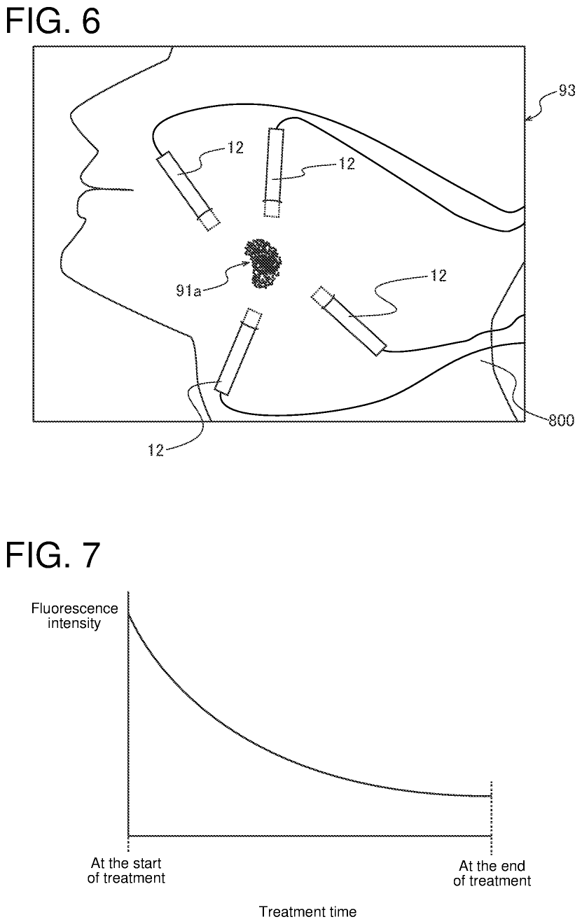

Further, the display unit 90 is configured to display a composite image 93 (see FIG. 6) in which the fluorescence distribution image 91 (see FIG. 4) and the visible light image 92 (see FIG. 5) are superimposed. With this, the user, such as a doctor, can simultaneously confirm the distribution 91a of fluorescence, the position of the therapeutic probe 12, and the position of the therapeutic light, which are displayed on the display unit 90. Note that the composite image 93 is generated by superimposing the image data of the fluorescence distribution image 91 and the visible light image 92 by the PC 70. Note that the fluorescence distribution image 91 and either the visible light image 92 or the composite image 93 may be simultaneously displayed side by side on the display unit 90. Further, the display unit 90 may be configured to display one of the fluorescence distribution image 91, the visible light image 92, and the composite image 93 by switching between them.

### (Change in Signal Waveform of Fluorescence)

The fluorescence detection unit 20 and the fluorescence imaging unit 31 detect the fluorescence emitted from the fluorescent substance 901 of the drug 900, as described above. However, when the fluorescent substance 901 of the drug 900 undergoes a photochemical reaction due to continued exposure to the therapeutic light, fluorescence will no longer be emitted from the drug 900 (fluorescent substance 901). Therefore, as shown in FIG. 7, the intensity of the detected fluorescence (fluorescence intensity) decreases (attenuates) in accordance with the increase in treatment time. Note that the change in the fluorescence intensity showing the change in accordance with the increase in treatment time, as shown in FIG. 7, is calculated based on the total fluorescence signal value (the area of the signal waveform) in the signal waveform of the fluorescence emitted from the fluorescent substance 901 in the drug 900.

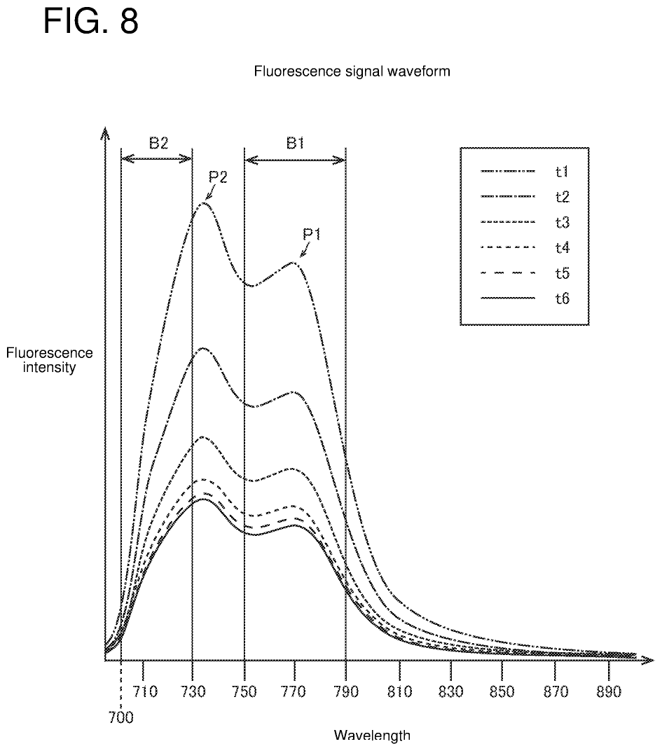

FIG. 8 shows the signal waveform (spectrum) of the fluorescence at each of the treatment times t1, t2, t3, t4, t5, and t6. Note that the treatment time is shorter in the order of the treatment times t1, t2, t3, t4, t5, and t6. As shown in FIG. 8, the overall magnitude of the fluorescence signal waveform decreases as the treatment time increases (in the order of treatment times t1, t2, t3, t4, t5, and t6). In other words, as the treatment time increases, the intensity (fluorescence intensity) of the fluorescence emitted from the fluorescent substance 901 of the drug 900 attenuates (decreases).

Further, as shown in FIG. 8, the signal waveform of the fluorescence emitted from the fluorescent substance 901 of the drug 900 excited by the therapeutic light has a plurality of peaks. Specifically, the signal waveform of the fluorescence emitted from the IRDye700 (registered trademark), which is the fluorescent substance 901 of the drug 900 excited by the therapeutic light, has two peaks (the peak P1 and the peak P2) at each of the treatment times t1, t2, t3, t4, t5, and t6.

The first wavelength band B1, which is the region in which the PC 70 selectively acquires signal information, is a wavelength band that includes the peak P1, which is located at 770 nm and the vicinity thereof, among a plurality of peaks in the signal waveform of the fluorescence emitted by the fluorescent substance 901 of the drug 900. Note that the peak P1 is one example of the “first peak” as recited in claims. Specifically, the PC 70 selectively acquires signal information (signal waveform) in the wavelength band of 750 nm or more and 790 nm or less, as the first wavelength band B1.

Further, the second wavelength band B2, which is the region in which the PC 70 selectively acquires signal information, is the wavelength band that includes the rising portion of the peak P2, which is located in a wavelength band with a wavelength shorter than 770 nm (the peak position of the peak P1), among the plurality of peaks in the signal waveform of the fluorescence emitted from the fluorescent substance 901 of the drug 900. Note that the peak P2 is one example of the “second peak” as recited in claims. Specifically, the PC 70 selectively acquires signal information in the wavelength band of 700 nm or more and 730 nm or less, as the second wavelength band B2. In other words, the PC 70 selectively acquires the signal information (signal waveform) in the second wavelength band B2, which is a wavelength band with a shorter wavelength than the first wavelength band B1 and includes a wavelength of 700 nm or more and 730 nm or less, from the signal waveform of the fluorescence emitted from the fluorescent substance 901 of the drug 900.

Further, the fluorescence imaging unit 31 detects the light (fluorescence) with a wavelength of 700 nm or more by the wavelength selectivity of the optical filter, as described above. With this, the fluorescence imaging unit 31 is configured to image the distribution of the fluorescence emitted from the fluorescent substance 901 of the drug 900 excited by the therapeutic light, based on the light in the wavelength bands that include the first wavelength band B1 (wavelength band of 750 nm or more and 790 nm or less) and the second wavelength band B2 (wavelength band of 700 nm or more and 730 nm or less).

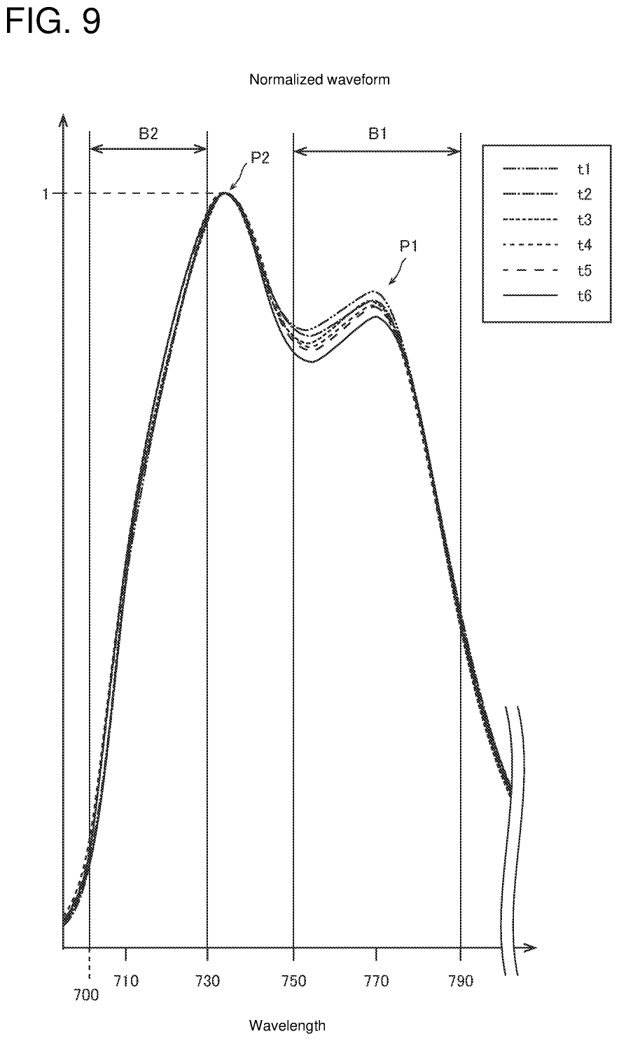

Further, FIG. 9 shows the waveforms which are obtained by normalizing the signal waveforms at each of the treatment times t1, t2, t3, t4, t5 and t6, with the value (the maximum value of each signal waveform) of each peak P2 set to 1. In the waveforms normalized based on the value of each peak P2 (the maximum value of each signal waveform), as shown in FIG. 9, in the first wavelength band B1, the signal waveform change (attenuation of the fluorescence intensity) in accordance with the increase in treatment time is larger than that in the other wavelength bands. In the second wavelength band B2, it can be confirmed that the signal waveform change (attenuation of the fluorescence signal strength) in accordance with the increase in treatment time is smaller than that in the other wavelength bands. In other words, in some wavelength bands (the first wavelength band B1 and the second wavelength band B2), a characteristic waveform change is observed in accordance with the increase in treatment time.

Specifically, as shown in FIG. 9, in the first wavelength band B1 including the peak P1, as the treatment time increases and the attenuation of the fluorescence strength increases, the falling width of the peak P1 with respect to the peak P2 (each maximum value of the signal waveforms) increases (the waveform becomes smaller), as shown in the normalized waveforms. Therefore, in the first wavelength band B1, the change in the signal waveform (the attenuation of the fluorescence strength) in accordance with the increase in the treatment time is larger than that in other wavelength bands.

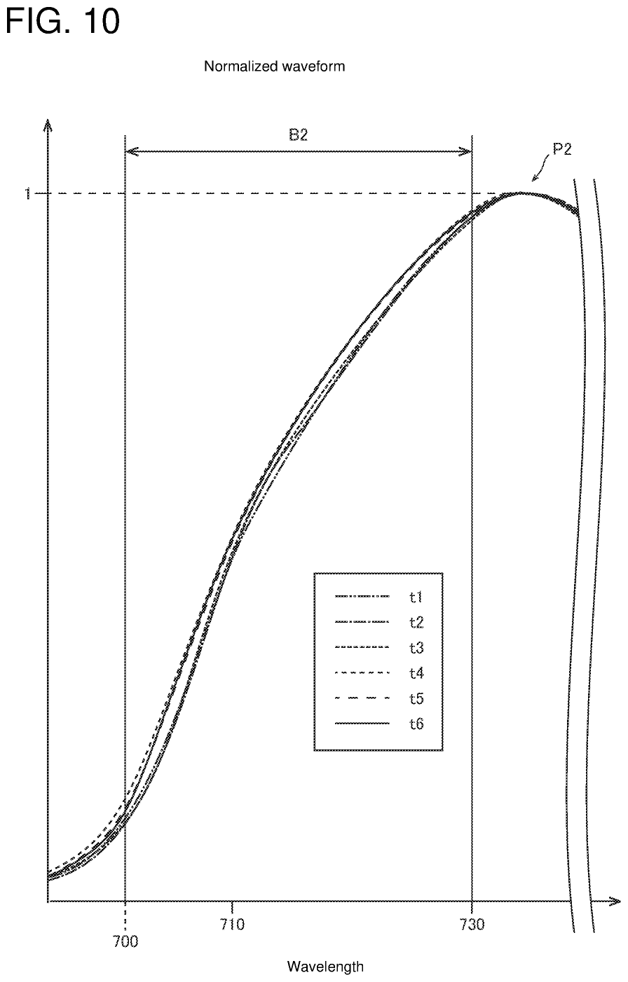

On the other hand, as shown in FIG. 10, in the second wavelength band B2, which is the rising portion of the peak P2, the result that there is a tendency for the bulge at the rising portion of the peak P2 to become larger (the waveform becomes larger) as the treatment time becomes longer and the attenuation of the fluorescence intensity increases, is shown in the normalized waveform. Therefore, in the second wavelength band B2, it can be seen that the change in the signal waveform (attenuation of the fluorescence signal strength) in accordance with the increase in the treatment time is smaller than that in the other wavelength bands and that a signal waveform change (change in the opposite direction) different from that in the first wavelength band B1 is occurring.

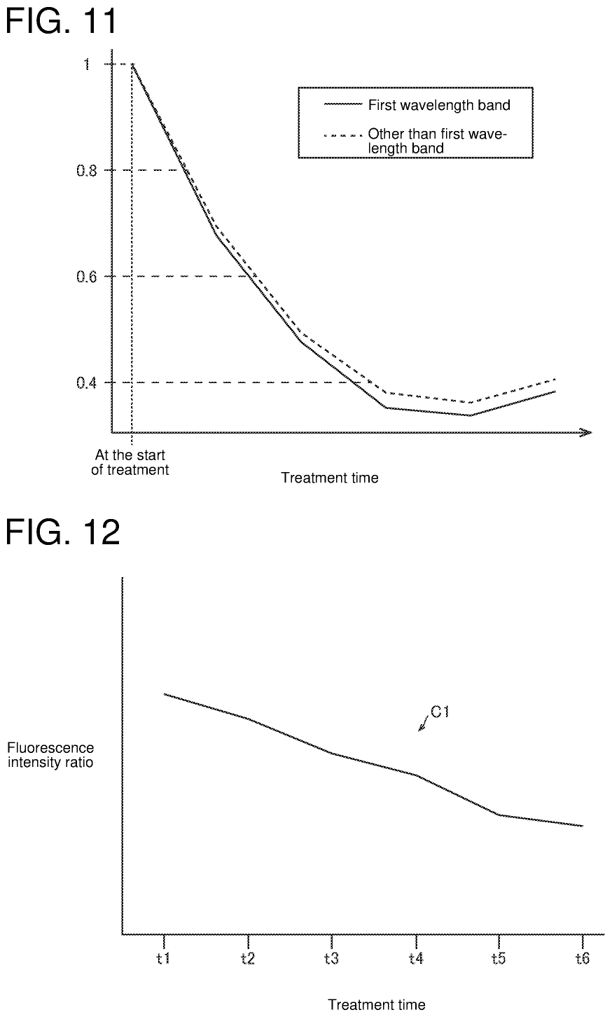

Further, FIG. 11 is a graph showing the relative change with respect to the fluorescence intensity (fluorescence signal value) at the start of treatment for each of the fluorescence intensity (fluorescence signal value) based on the light in the first wavelength band B1 and the fluorescence intensity (fluorescence signal value) based on the light in a wavelength band other than the first wavelength band B1, with the start of treatment set at the start of treatment as 1.

Then, as shown in FIG. 11, when comparing the relative change in accordance with the increase in the treatment time of the fluorescence intensity based on the light in the first wavelength band B1 with the relative change in accordance with the increase in the treatment time of the fluorescence intensity based on the light in the wavelength bands other than the first wavelength band B1, the fluorescence intensity based on the light in the wavelength band of the first wavelength band B1 has a larger change in accordance with the increase in the treatment time. Therefore, by acquiring (confirming) the progress of the treatment based on the light in the first wavelength band B1, the information indicating the progress of the treatment can be acquired more significantly than when acquiring (confirming) the progress of the treatment based on the entire fluorescence emitted from the fluorescent substance 901 of the drug 900, including in the wavelength bands other than the first wavelength band B1.

Further, in this embodiment, the fluorescence detection unit 20 is configured to acquire (detect) the signal waveform of the fluorescence emitted from the fluorescent substance 901 of the drug 900 by sequentially scanning (scanning) the fluorescence emitted from the fluorescent substance 901 of the drug 900 in each predetermined wavelength band. Specifically, the fluorescence detection unit 20 sequentially scans the signal waveforms in the wavelength band of about 690 nm to about 900 nm among the signal waveforms of the fluorescence emitted from the fluorescent substance 901 of the drug 900, for each predetermined wavelength band. Note that the time interval (scan speed) for acquiring the signal waveform (light in the wavelength band of about 690 nm to about 900 nm) of the fluorescence emitted from the fluorescent substance 901 of the drug 900 is configured to be changeable by the user, for example, at 0.5 second intervals, 1 second intervals, and 2 seconds intervals. Further, it may be configured such that the wavelength band of the light (signal waveform) acquired by the fluorescence detection unit 20 can be changed by the user from about 690 nm to about 900 nm.

Further, in this embodiment, the fluorescence detection unit 20 is configured to acquire (detect) the signal waveform of the fluorescence emitted from the fluorescent substance 901 of the drug 900 by sequentially scanning (scanning) the fluorescence emitted from the fluorescent substance 901 of the drug 900 in each predetermined wavelength band. Further, the fluorescence detection unit 20 is configured to sequentially scan the fluorescence emitted from the fluorescent substance 901 of the drug 900 in each predetermined wavelength band to detect the signal waveform of the light in the second wavelength band B2 from the fluorescence emitted from the fluorescent substance 901 of the drug 900.

In other words, the fluorescence detection unit 20 is configured to sequentially scan the fluorescence emitted from the fluorescent substance 901 of the drug 900 in each predetermined wavelength band to detect each of the signal waveforms of the light in the first wavelength band B1 and the light in the second wavelength band B2 from the fluorescence emitted from the fluorescent substance 901 of the drug 900.

In this embodiment, the PC 70 is configured to selectively acquire the signal waveform of the light in the first wavelength band B1, which corresponds to the light in the first wavelength band B1, as the first signal information corresponding to the light in the first wavelength band B1, which is a wavelength band that includes a wavelength at or around 770 nm among the signal waveforms of the fluorescence emitted from the substance 901 of the drug 900 detected by the fluorescence detection unit 20.

Further, Further, the PC 70 is configured to generate first fluorescence change information C1 (see FIG. 12), which is information on the change in the fluorescence intensity in the first wavelength band B1, based on the acquired signal waveform (first signal information) of the light in the first wavelength band B1. Specifically, the PC 70 is configured to generate the first fluorescence change information C1 based on the change in the signal waveform due to the increase in the irradiation time of the therapeutic light, in the first wavelength band B1 of the signal waveform of the fluorescence emitted from the fluorescent substance 901 of the drug 900.

In this embodiment, the PC 70 acquires the signal waveform (first signal information) of the light in the first wavelength band B1 detected by the fluorescence detection unit 20 by sequentially scanning each predetermined wavelength band. In other words, the PC 70 is configured to generate the first fluorescence change information C1 based on the signal waveform of the light in the first wavelength band B1 detected by the fluorescence detection unit 20 by sequentially scanning each predetermined wavelength band.

In this embodiment, as shown in FIG. 12, the first fluorescence change information C1 shows the ratio of the magnitude of the signal waveform in the first wavelength band B1 after normalization shown in FIG. 9 with respect to the magnitude of the signal waveform in the first wavelength band B1 at the start of treatment for each treatment time. The normalized signal waveform in the first wavelength band B1 decreases in the waveform in accordance with the increase in the treatment time, as described above (see FIG. 9). Therefore, as shown in FIG. 12, the relative change in the fluorescence intensity (fluorescence signal value) based on the light in the first wavelength band B1 with the increase in the temporal change in the treatment time is a rightward downward change.

Further, in this embodiment, the PC 70 is configured to selectively acquire the signal waveform of the light in the second wavelength band B2 (the second signal information corresponding to the light in the second wavelength band B2) in addition to the signal waveform of the light in the first wavelength band B1 (the first signal information corresponding to the light in the first wavelength band B1).

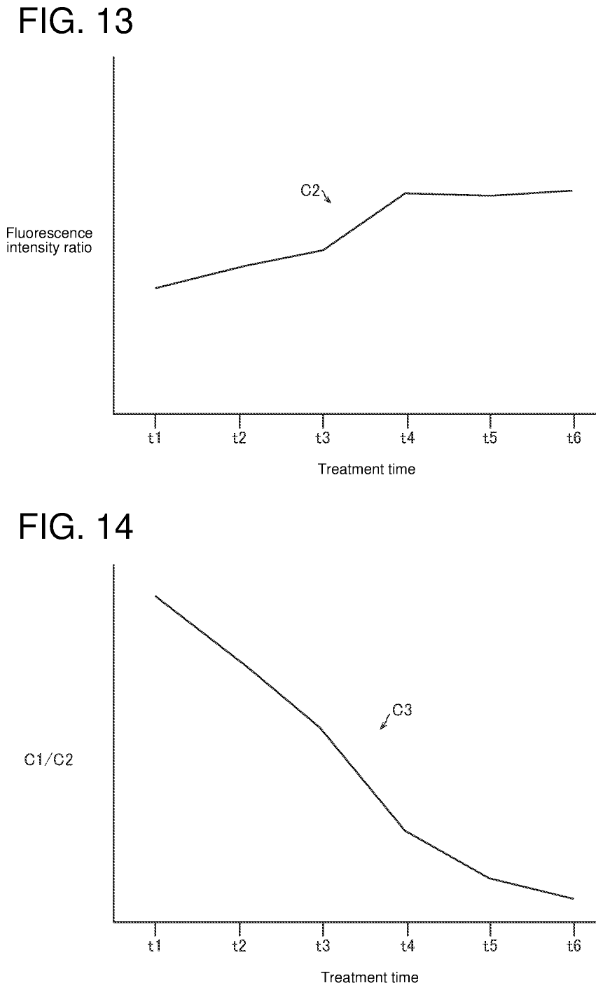

The PC 70 is configured to generate the second fluorescence change information C2 (see FIG. 13), which is information on the fluorescence intensity change in the second wavelength band B2, based on the acquired signal waveform of the light in the second wavelength band B2 (second signal information). Specifically, the PC 70 is configured to generate the second fluorescence change information C2 based on the signal waveform change in the second wavelength band B2 of the signal waveform in accordance with the increase in the irradiation time of the therapeutic light, in the second wavelength band B2 of the signal waveform of the fluorescence emitted from the fluorescent substance 901 of the drug 900.

In this embodiment, the PC 70 acquires the signal waveform (second signal information) of the light in the second wavelength band B2 detected by the fluorescence detection unit 20 by sequentially scanning each predetermined wavelength band. In other words, the PC 70 is configured to generate the second fluorescence change information C2 based on the signal waveform of the light in the second wavelength band B2 detected by the fluorescence detection unit 20 by sequentially scanning each predetermined wavelength band.

In this embodiment, as shown in FIG. 13, the second fluorescence change information C2 shows the ratio of the magnitude of the signal waveform in the second wavelength band B2 after normalization shown in FIG. 9 to the magnitude of the signal waveform in the second wavelength band B2 at the start of treatment for each treatment time. The normalized signal waveform in the second wavelength band B2 tends to swell in the waveform in accordance with the increase in the treatment time, as described above (see FIG. 9). Therefore, as shown in FIG. 13, the relative change in the fluorescence intensity (fluorescence signal value) in accordance with the increase in the treatment time based on the light in the second wavelength band B2 becomes a rightward upward change.

The PC 70 is configured to generate a treatment progress index C3 (see FIG. 14), which is an index of the treatment progress based on the first fluorescence change information C1 and the second fluorescence change information C2. Specifically, the PC 70 is configured to calculate the treatment progress index C3 based on the ratio of the first fluorescence change information C1 to the second fluorescence change information C2.

In this embodiment, the PC 70 calculates the ratio of the first fluorescence change information C1 to the second fluorescence change information C2 (first fluorescence change information C1/second fluorescence change information C2) as the treatment progress index C3. In other words, the PC 70 calculates the ratio of the first fluorescence change information C1, which is generated based on the signal waveform of the light in the first wavelength band B1 whose signal waveform changes significantly in accordance with the increase in the treatment time, to the second fluorescence change information C2, which is generated based on the signal waveform of the light in the second wavelength band B2 in which the change (change in the opposite direction) of the signal waveform, which is different from that in the first wavelength band B1, occurs in accordance with the increase in the treatment time. With this, the change with the increase in the treatment time becomes larger (the slope increases) as compared with the first fluorescence change information C1, and therefore, it is possible to more significantly acquire (confirm) the progress of treatment based on the change in the fluorescence signal waveform (attenuation of the fluorescence signal). In the treatment support device 100, by quantifying the changes in the signal waveform with the increase in the treatment time as described above, it is possible to provide new information (indexes) different from the attenuation of the fluorescence intensity, such as the first fluorescence change information C1, the second fluorescence change information C2, and the treatment progress index C3. Since the attenuation of the fluorescence intensity varies from patient 800 to patient 800, this new information (index), which is different from the attenuation of the fluorescence intensity, is especially useful when it is difficult to determine the progress of treatment only based on the attenuation of fluorescence intensity.

### (Display by Display Unit)

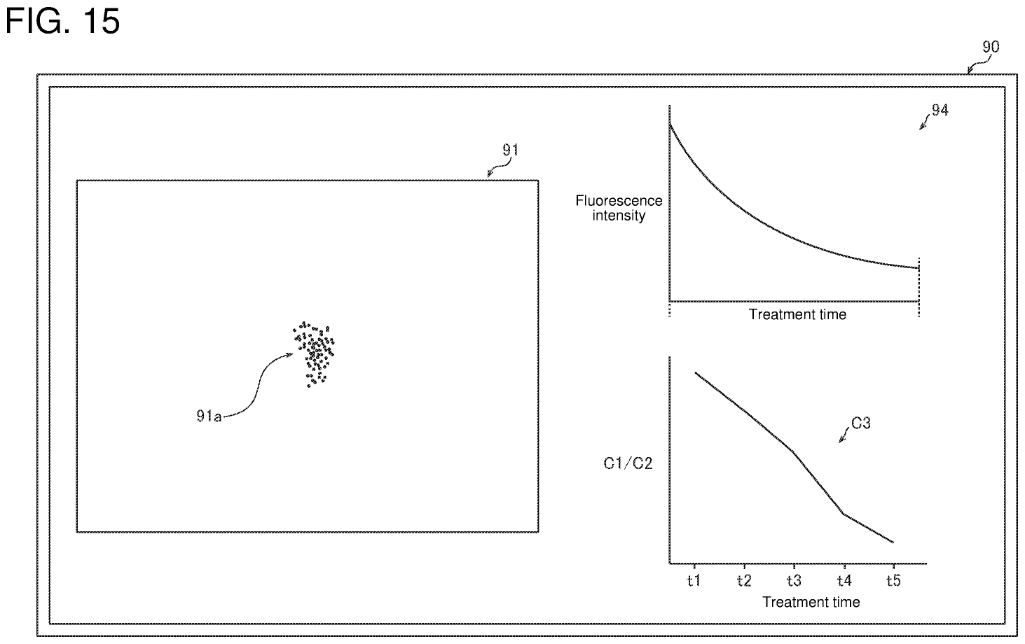

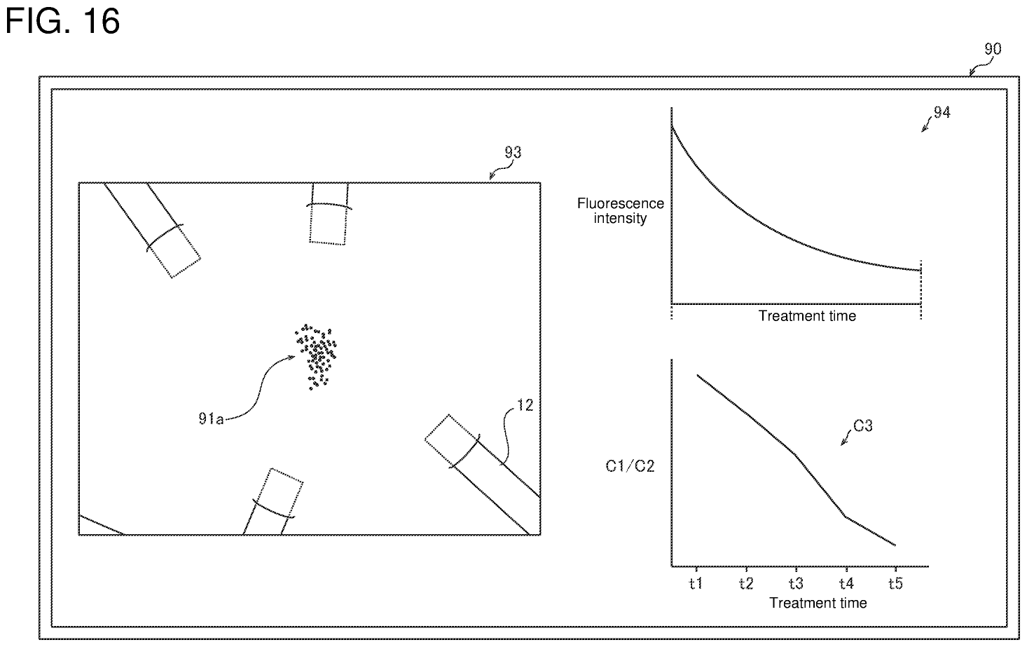

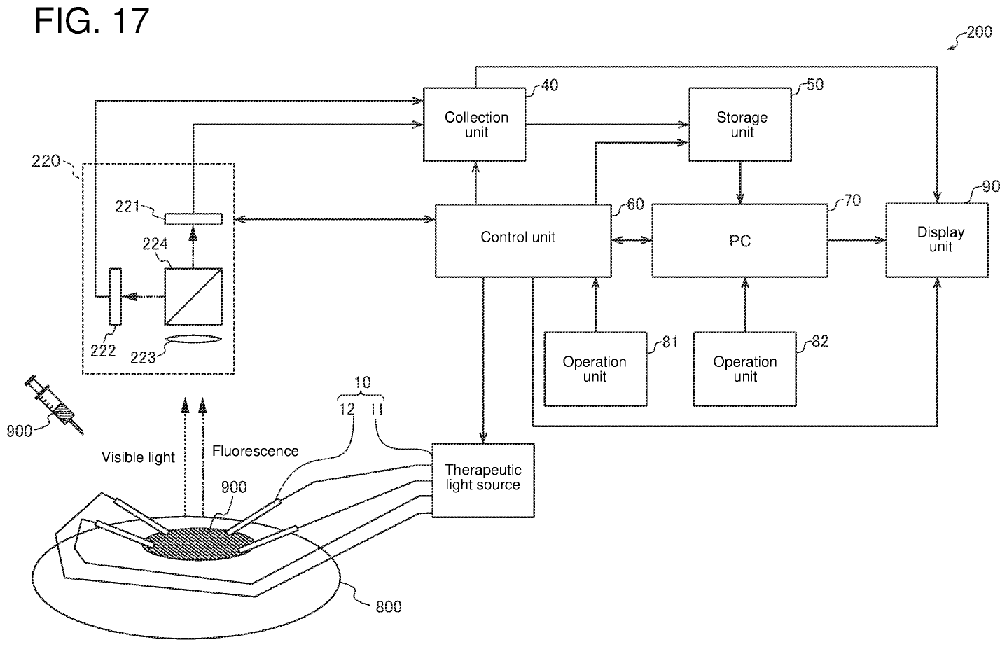

As shown in FIG. 15 and FIG. 16, the display unit 90 is configured to notify the progress of the treatment based on the irradiation of the therapeutic light, according to the first fluorescence change information C1. In this embodiment, the display unit 90 is configured to inform the user of the progress of the treatment based on the irradiation of the therapeutic light by displaying the treatment progress index C3, which is calculated based on the ratio of the first fluorescence change information C1 to the second fluorescence change information C2.

In this embodiment, as shown in FIG. 15, the display unit 90 is configured to display a treatment progress index C3, which is an index of the treatment progress based on the first fluorescence change information C1 and the second fluorescence change information C2, and a fluorescence distribution image 91, which is an image showing the distribution of the fluorescence imaged by the fluorescence imaging unit 31. Further, the display unit 90 is configured to display a fluorescence attenuation image 94 showing the change in fluorescence intensity in accordance with the increase in the treatment time, along with the treatment progress index C3 and the fluorescence distribution image 91. The fluorescence attenuation image 94 shows the change in the total value (the area of the signal waveform) of the fluorescence intensity in the fluorescence signal waveform, as described above. Further, the treatment support device 100 is configured to notify the user that the value (the value based on the first fluorescence change information C1) of the treatment progress index C3 or the value of the total fluorescence intensity (the area of the signal waveform) in the signal waveform of the fluorescence detected by the fluorescence detection unit 20 exceeds a preset threshold value, for example, by changing the display color of each value, by displaying a message, or by emitting a sound.

Further, the treatment support device 100 is configured to switch between images displayed on the display unit 90. Specifically, the display unit 90 can display the treatment progress index C3 and the composite image 93, as shown in FIG. 16. Further, the display unit 90 can display a fluorescence attenuation image 94 showing the change in the fluorescence intensity in accordance with the increase in the treatment time, along with the treatment progress index C3 and the composite image 93.

### Effects of this Embodiment

In this embodiment, the following effects can be obtained.

In this embodiment, as described above, the PC 70 (change information generation unit) selectively acquires the signal waveform (first signal information corresponding to the light in the first wavelength band B1) of the light in the first wavelength band B1, which is a wavelength band that includes a wavelength of approximately 770 nm. The PC 70 generates the first fluorescence change information C1, which is information on the change in the fluorescence strength in the first wavelength band B1, based on the acquired signal waveform of the light in the first wavelength band B1 (first signal information). With this, the information (the first fluorescence change information C1) on the change in the fluorescence intensity in the wavelength band at 770 nm and the vicinity thereof, where the change in the signal waveform of fluorescence in accordance with the increase in the treatment time is found to be larger than that in other wavelength bands, is selectively acquired. This excludes the information on the change in fluorescence intensity in the signal waveform of the fluorescence emitted from the fluorescent substance of the drug, where the change in the signal waveform of the fluorescence caused by the increase in treatment time is small. As a result, as compared with the case of acquiring the information on the change in the fluorescence intensity of the entire waveform of the fluorescence emitted from the fluorescent substance of the drug, it is possible to acquire the change in the fluorescence signal waveform due to the increase in the treatment time with high sensitivity. With this, it is possible to acquire the change in the signal waveform of the fluorescence due to the increase in the treatment time with high sensitivity, thereby acquiring the treatment progress of photoimmunotherapy with high sensitivity. Furthermore, based on the first fluorescence change information C1 reflecting the change in the fluorescence signal waveform due to the increase in the treatment time, the display unit 90 (notification unit) displays the treatment progress. With this, the user, such as a doctor, can grasp the progress of the treatment acquired with high sensitivity by the display unit 90.

In addition, in the treatment support device 100 according to the above-described embodiment, the following further effects can be obtained by configuring the device as follows.

In this embodiment, as described above, the first wavelength band B1 is a wavelength band that includes the peak P1 (first peak) located at 770 nm or in the vicinity thereof among the plurality of peaks in the signal waveform of the fluorescence emitted from the fluorescent substance 901 of the drug 900. With this, compared with the other wavelength bands, it is possible to acquire the information (the first fluorescence change information C1) on the change in the fluorescence intensity at the peak P1 located in the wavelength band at or around 770 nm, where the change in the signal waveform of the fluorescence due to the increase in the treatment time is found to be larger than in the other wavelength bands. As a result, the information on the portion of the peak P1, where the signal waveform changes significantly due to the increase in the treatment time, is selectively acquired, so that the change in the signal waveform due to the increase in the treatment time can be acquired more sensitively than when acquiring information on the entire signal waveform of the fluorescence emitted from the fluorescent substance 901 in the drug 900.

Further, in this embodiment, as described above, the PC 70 (change information generation unit) is configured to generate the first fluorescence change information C1 based on the change in the signal waveform in the first wavelength band B1 of the signal waveform of the fluorescence emitted from the fluorescent substance 901 of the drug 900 due to the increase in the irradiation time of the therapeutic light. With this, it is possible to easily confirm the progress of the treatment due to the increase in the treatment time, by the first fluorescence change information C1, which is based on the change in the signal waveform due to the increase in the irradiation time of the therapeutic light.

Further, in this embodiment, as described above, the fluorescence detection unit 20 is configured to detect the signal waveform of the light in the first wavelength band B1 from the fluorescence emitted from the fluorescent substance 901 of the drug 900 by sequentially scanning the fluorescence emitted from the fluorescent substance 901 of the drug 900 in each predetermined wavelength band. With this, the fluorescence detection unit 20 can acquire the signal waveform of the light in a wavelength band other than the first wavelength band B1 as well as the signal waveform of the light in the first wavelength band B1 by sequentially scanning the fluorescence emitted from the fluorescent substance 901 of the drug 900 in each predetermined wavelength band. As a result, unlike the case in which the fluorescence detection unit 20 detects only the signal waveform of the light in the first wavelength band B1, it is not necessary to provide a detection unit separately to acquire the signal waveform in a wider wavelength band than the first wavelength band B1, such as the entire signal waveform of the fluorescence emitted from the fluorescent substance 901 of the drug 900. Therefore, it is possible to suppress the increase in the number of parts and the complexity of the device configuration. Further, the PC 70 (change information generation unit) is configured to generate the first fluorescence change information C1 based on the signal waveform of the light in the first wavelength band B1 detected by the fluorescence detection unit 20 by sequentially scanning each predetermined wavelength band. With this, unlike the case of acquiring signal waveforms of light detected selectively by an optical filter or other means, the PC 70 can easily change the wavelength band of the signal waveform to be selectively acquired to generate the first fluorescence change information C1. As a result, even in cases where noise occurs in a part of the wavelength band from which the signal waveform is selectively acquired, the band of the noisy part can be easily removed. Thus, the first fluorescence change information C1 can be generated with high accuracy.

Further, in this embodiment, as described above, in addition to the signal waveform (the first signal information corresponding to the light in the first wavelength band B1), the PC 70 (change information generation unit) is configured to selectively acquire, among the signal waveforms of the light emitted from the fluorescent substance 901 of the drug 900, the signal waveform (the second signal information corresponding to the light in the second wavelength band B2) of the light in a wavelength band shorter in wavelength than the first wavelength band B1 of 700 nm or more and 730 nm or less. The PC 70 is configured to generate the second fluorescence change information C2, which is information on the change in the fluorescence intensity in the second wavelength band B2, based on the acquired signal waveform of the light in the second wavelength band B2. Further, the PC 70 is configured to generate the treatment progress index C3, which is an index of the treatment progress based on the first fluorescence change information C1 and the second fluorescence change information C2. With this, based on both the first fluorescence change information C1 and the second fluorescence change information C2, i.e., the first wavelength band B1 where the signal waveform change due to the increase in the treatment time was found to be larger than in other wavelength bands, and the second wavelength band B2 where the signal waveform change due to the increase in the treatment time was found to be smaller than in other wavelength bands, the treatment progress index C3 is generated. As a result, compared with the case in which the treatment progress index C3 is generated based only on the first fluorescence change information C1, the characteristics of the changes in the signal waveform due to the increase in the treatment time can be reflected by the treatment progress index C3. With this, the change in the signal waveform due to the increase in the treatment time can be acquired with high sensitivity, and thus the progress of the treatment by photoimmunotherapy can be acquired with higher sensitivity.

Further, in this embodiment, as described above, the second wavelength band B2 is a wavelength band that includes the rising portion of the peak P2 (the second peak), which is located in a wavelength band shorter in wavelength than 770 nm, among a plurality of peaks in the signal waveform of the fluorescence emitted from the fluorescent substance 901 of the drug 900. With this, it is possible to acquire the information (the second fluorescence change information C2) on the change in the fluorescence intensity including the rising portion of the peak P2, where the change in the signal waveform of the fluorescence due to the increase in the treatment time is found to be smaller than in the other wavelength bands. As a result, the information on the rising part of the peak P2, where the change in the signal waveform due to the increase in the treatment time is characteristic compared with other wavelength bands, is selectively acquired. Therefore, compared with the case of acquiring the information on the entire signal waveform of the fluorescence emitted from the fluorescent substance 901 of the drug 900 due to the increase in the treatment time, the change in the signal waveform due to the increase in the treatment time is acquired with higher sensitivity.

Further, in this embodiment, as described above, the PC 70 (change information generation unit) is configured to generate the second fluorescence change information C2 based on the change in the signal waveform in the second wavelength band B2 of the signal waveform of the fluorescence emitted from the fluorescent substance 901 of the drug 900 due to the increase in the irradiation time of the therapeutic light. With this, it is possible to easily confirm the progress of the treatment due to the increase in the treatment time by the second fluorescence change information C2 based on the change in the signal waveform due to the increase in the irradiation time of the therapeutic light.

Further, in this embodiment, as described above, the PC 70 (change information generation unit) is configured to calculate the treatment progress index C3 based on the ratio of the first fluorescence change information C1 to the second fluorescence change information C2. With this, by calculating the ratio of the first fluorescence change information C1, which is generated based on the signal waveform of light in the first wavelength band B1, where the change in the signal waveform due to the increase in the treatment time is larger, to the second fluorescence change information C2, which is generated based on the signal waveform of light in the second wavelength band B2, where the change in the signal waveform due to the increase in the treatment time occurs in a different direction (opposite to the first wavelength band B1), it is possible to acquire the change in the signal waveform of fluorescence more significantly. As a result, the progress of the treatment can be more prominently shown by the treatment progress index C3.

Further, in this embodiment, as described above, the fluorescence detection unit 20 is configured to detect each of the signal waveforms of the light in the first wavelength band B1 and the light in the second wavelength band B2 from the fluorescence emitted from the fluorescent substance 901 of the drug 900 by sequentially scanning the fluorescence emitted from the fluorescent substance 901 of the drug 900 in each predetermined wavelength band. With this, the fluorescence detection unit 20 can acquire the signal waveform of the light in a wavelength band other than the first wavelength band B1 and the second wavelength band B2, as well as the signal waveform of the light in the first wavelength band B1 and the second wavelength band B2, by sequentially scanning the fluorescence emitted from the fluorescent substance 901 of the drug 900 in each predetermined wavelength band. As a result, unlike the case in which the fluorescence detection unit 20 detects only the signal waveform of the light in the first wavelength band B1 and the signal waveform of the light in the second wavelength band B2, it is not necessary to separately provide a detection unit to acquire the signal waveform of a wavelength band wider than the first wavelength band B1 and the second wavelength band B2, such as the entire signal waveform of the fluorescence emitted from the substance 901 of the drug 900. Therefore, it is possible to suppress the increase in the number of parts and the complexity of the device configuration. Further, the PC 70 (change information generation unit) is configured to generate the second fluorescence change information C2 based on the signal waveform of the light in the second wavelength band B2 detected by the fluorescence detection unit 20 by sequentially scanning each predetermined wavelength band. With this, unlike the case of acquiring the signal waveform of the light detected selectively by an optical filter or other means, the PC 70 can easily change the wavelength band of the signal waveform to be selectively acquired to generate the second fluorescence change information C2. As a result, even in cases where noise occurs in a part of the wavelength band from which the signal waveform is selectively acquired, the band of the noisy part can be easily removed. Thus, the second fluorescence change information C2 can be generated with high accuracy.

Further, in this embodiment, as described above, the fluorescence imaging unit 31 is provided separately from the fluorescence detection unit 20 and is configured to image the distribution of the fluorescence emitted from the fluorescent substance 901 of the drug 900 excited by the therapeutic light, based on the light in the wavelength bands including the first wavelength band B1 and the second wavelength band B2. And, the display unit 90 (notification unit) is configured to display the treatment progress index C3, which is an index of the progress of the treatment based on the first fluorescence change information C1 and the second fluorescence change information C2. With this, the user can easily confirm the progress of the treatment by visually viewing the treatment progress index C3 displayed on the display unit 90. Further, the display unit 90 (notification unit) is configured to display the fluorescence distribution image 91, which is an image showing the distribution of the fluorescence imaged by the fluorescence imaging unit 31. With this, the user can easily confirm the accumulation level of the drug 900 (the fluorescent substance 901) from the fluorescence distribution by visually viewing the fluorescence distribution image 91 displayed on the display unit 90.

### Modifications

Note that the embodiments disclosed here should be considered illustrative and not restrictive in all respects. It should be noted that the scope of the invention is indicated by claims and is intended to include all modifications (modified examples) within the meaning and scope of the claims and equivalents.

For example, in the above-described embodiment, an example is shown in which the treatment support device 100 displays the treatment progress index C3 based on the first fluorescence change information C1 on the display unit 90 (notification unit), but the present invention is not limited thereto. In the present invention, the treatment support device may be configured such that a threshold is preset for the first fluorescence change information or the value of the treatment progress index and that the notification unit provides an acoustic notification when the first fluorescence change information or the treatment progress index value exceeds the set threshold.

Further, in the above-described embodiment, an example is shown in which the first wavelength band B1 is a wavelength band (about 750 nm to about 790 nm) that includes the peak P1 (first peak) located at or around 770 nm among the plurality of peaks in the signal waveform of the fluorescence emitted from the fluorescent substance 901 of the drug 900, but the present invention is not limited thereto. In the present invention, the first wavelength band may be a wavelength band that includes only a part of the first peak.

Further, in the above-described embodiment, an example is shown in which the second wavelength band B2 is a wavelength band (700 nm or more and 730 nm or less) that includes the rising portion of the peak P2 (the second peak), which is located in a wavelength band with a wavelength shorter than 770 nm, among the plurality of the peaks in the signal waveform of the fluorescence emitted from the fluorescent substance 901 of the drug 900, but the present invention is not limited thereto. In the present invention, the second wavelength band may include the top and the falling portion of the second peak.

Further, in the above-described embodiment, an example is shown in which the PC 70 (change information generation unit) is configured to generate the first fluorescence change information C1, based on the change in the signal waveform in the first wavelength band B1 of the signal waveform of the fluorescence emitted from the fluorescent substance 901 of the drug 900 due to the increase in the irradiation time of the therapeutic light, but the present invention is not limited thereto. In the present invention, the ratio of the maximum value of the fluorescence intensity in the first wavelength band to the maximum value of the fluorescence intensity in the entire signal waveform of the fluorescence emitted from the fluorescent substance of the drug may be generated as the first fluorescence change information. In this case, it may be configured such that a threshold value may be set for the ratio of the maximum value of the fluorescence intensity in the first wavelength band to the maximum value of the fluorescence intensity in the entire signal waveform of the fluorescence emitted from the fluorescent substance of the drug, and that the notification unit performs a notification by sound or an image when the ratio exceeds the set threshold.

Further, in the above-described embodiment, an example is shown in which the PC 70 (change information generation unit) is configured to generate the second fluorescence change information C2, based on the change in the signal waveform in the second wavelength band B2 of the signal waveform of the fluorescence emitted from the fluorescent substance 901 of the drug 900 due to the increase in the irradiation time of the therapeutic light, but the present invention is not limited thereto. In the present invention, the ratio of the maximum value of the fluorescence intensity in the second wavelength band to the maximum value of the fluorescence intensity in the entire signal waveform of the fluorescence emitted from the fluorescent substance of the drug may be generated as the second fluorescence change information.

Further, in the above-described embodiment, an example is shown in which the PC 70 (change information generation unit) generates the treatment progress index C3, which is an index of the treatment progress based on the first fluorescence change information C1 and the second fluorescence change information C2, but the present invention is not limited thereto. In the present invention, it may be configured such that only the first fluorescence change information is generated without generating a treatment progress index. In this case, the first fluorescence change information is used for display by the display unit (notification unit). Further, it may be configured such that only the first and second fluorescence change information are generated without generating a treatment progress index. In this case, the first fluorescence change information and the second fluorescence change information are used for display by the display unit (notification by the notification unit).

Further, in the above-described embodiment, an example is shown in which the PC 70 (change information generation unit) calculates the treatment progress index C3, based on the ratio of the first fluorescence change information C1 to the second fluorescence change information C2, but the present invention is not limited thereto. In the present invention, it may be configured such that the treatment progress index is calculated based on the difference between the first fluorescence change information and the second fluorescence change information.

Further, in the above-described embodiment, an example is shown in which the fluorescence detection unit 20 is configured to detect the signal waveform of the light in the first wavelength band B1 from the fluorescence emitted from the fluorescent substance 901 of the drug 900 by sequentially scanning the fluorescence emitted from the fluorescent substance 901 of the drug 900 for each predetermined wavelength band, but the present invention is not limited thereto. In the present invention, as in the treatment support device 200 according to a first modification shown in FIG. 17, it may be configured such that the fluorescence detection unit 220 separates the light in the first wavelength band out of the light transmitted through the lens 223 by the prism 224 and detects the separated light in the first wavelength band by the detection unit 221.

Further, in the above-described embodiment, an example is shown in which the PC 70 (change information generation unit) acquires the signal waveform of the light in the first wavelength band B1 as the first signal information, but the present invention is not limited thereto. In the present invention, it may be configured such that only a part of the signal waveform of the light in the first wavelength band is acquired as the first signal information. Further, it may be configured such that the maximum value of the fluorescence intensity in the first wavelength band, the minimum value of the fluorescence intensity in the first wavelength band, or the average value of the fluorescence intensity in the first wavelength band is acquired as the first signal information.

Further, in the above-described embodiment, an example is shown in which the fluorescence detection unit 20 is configured to detect each of the signal waveform of the light in the first wavelength band B1 and the signal waveform of the light in the second wavelength band B2 from the fluorescence emitted from the fluorescent substance 901 of the drug 900 by sequentially scanning the fluorescence emitted from the fluorescent substance 901 of the drug 900 for each predetermined wavelength band, but the present invention is not limited thereto. In the present invention, as in the treatment support device 200 according to the first modification shown in FIG. 17, it may be configured such that the fluorescence detection unit 220 separates the light in the second wavelength band out of the light transmitted through the lens 223 by the prism 224 and detects the separated light in the second wavelength band by the detection unit 222.

Further, in the above-described embodiment, an example is shown in which the PC 70 (change information generation unit) acquires the signal waveform of the light in the second wavelength band B2 as the second signal information, but the present invention is not limited thereto. In the present invention, it may be configured such that only a part of the signal waveform of the light in the second wavelength band is acquired as the second signal information. Further, it may be configured such that the maximum value of the fluorescence intensity in the second wavelength band, the minimum value of the fluorescence intensity in the second wavelength band, or the average value of the fluorescence intensity in the second wavelength band is acquired as the second signal information.

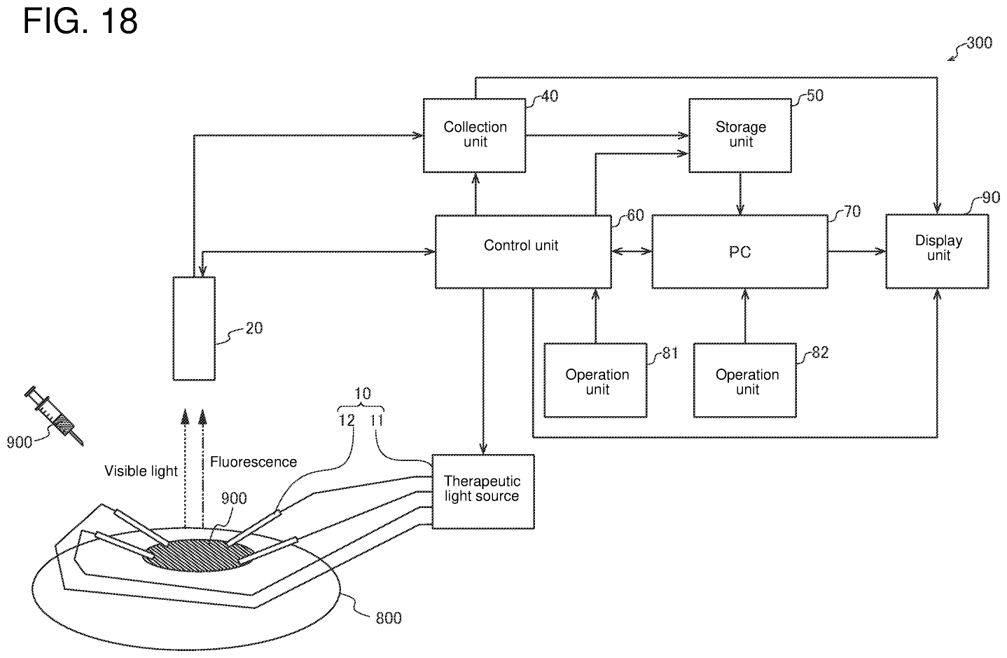

Further, in the above-described embodiment, an example is shown in which it is provided with the fluorescence imaging unit 31 that images the distribution of the fluorescence emitted from the fluorescent substance 901 of the drug 900 excited by the therapeutic light, but the present invention is not limited thereto. In the present invention, as in the treatment support device 300 according to a second modification shown in FIG. 18, the device may be equipped with only a fluorescence detection unit 20 that detects the fluorescence emitted from the fluorescent substance 901 of the drug 900 excited by the therapeutic light, without providing a fluorescence imaging unit that images the distribution of the fluorescence. In other words, the treatment support device does not need to acquire the distribution of the fluorescence.

Further, in the above-described embodiment, an example is shown in which the fluorescence imaging unit 31 is provided separately from the fluorescence detection unit 20, but the present invention is not limited thereto. In the present invention, the fluorescence imaging unit and the fluorescence detection unit may be constructed as a single unit.

Further, in the above-described embodiment, an example is shown in which the display unit 90 is configured to display the treatment progress index C3, which is an index of the treatment progress based on the first fluorescence change information C1 and the second fluorescence change information C2, and the fluorescence distribution image 91, which is an image showing the distribution of the fluorescence imaged by the fluorescence imaging unit 31 However, the present invention is not limited thereto. In the present invention, only the treatment progress index, which is an index of the progress of the treatment based on the first fluorescence change information and the second fluorescence change information, may be displayed on the display unit. In this case, the user can easily confirm the progress of the treatment by viewing the treatment progress index displayed on the display unit. Further, in the present invention, it may be configured such that only the fluorescence distribution image, which is an image showing the distribution of the fluorescence imaged by the fluorescence imaging unit, is displayed on the display unit, and the progress of the treatment based on the irradiation of the therapeutic light is notified based on the first fluorescence change information by a sound emitted by the notification unit. In this case, the user can easily confirm the degree of the drug accumulation from the fluorescence distribution by viewing the fluorescence distribution image displayed on the display unit, and can also easily confirm the progress of the treatment by the sound emitted by the notification unit.

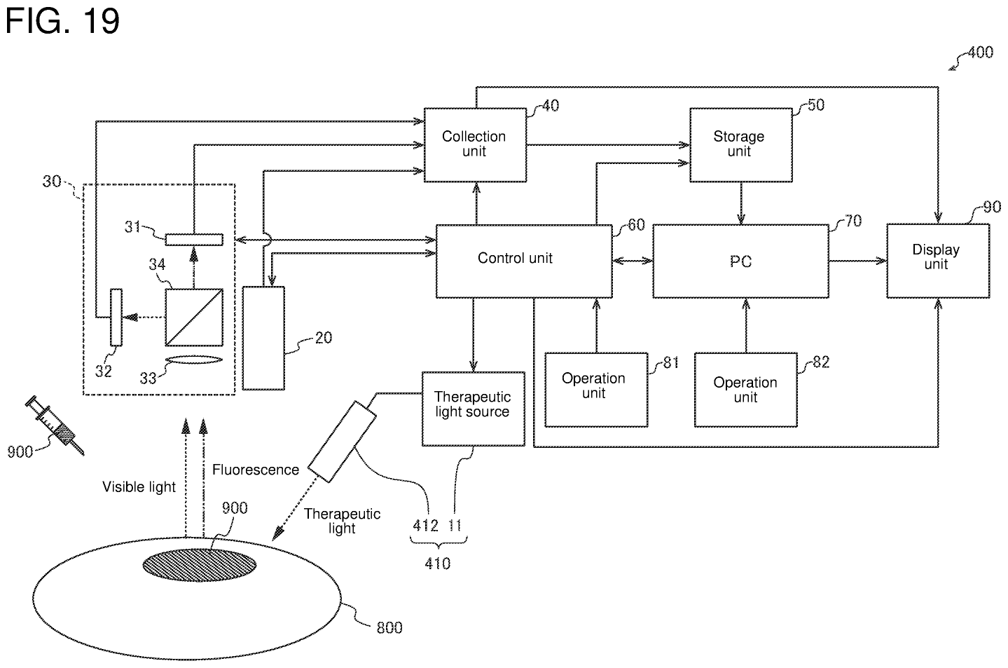

Further, in the above-described embodiment, an example is shown in which the therapeutic light (excitation light) is emitted from the therapeutic probe 12 (see FIG. 3) inserted into the body of the patient (subject) 800, the present invention is not limited thereto. In the present invention, as in the treatment support device 400 according to a third modification shown in FIG. 19, it may be configured such that the irradiation unit 410 is provided with an irradiation unit 412 for irradiating the therapeutic light (excitation light) from outside the body of the patient (subject) 800, and the irradiation unit 412 emits the therapeutic light (excitation light) from outside the body of the patient (subject) 800. Further, the treatment support device may include both the therapeutic probe 12 (see FIG. 3) and the irradiation unit 412 (see FIG. 19) as the irradiation unit.

Further, in the above-described embodiment, an example is shown in which the treatment support device 100 is provided with an irradiation unit 10 that emits therapeutic light (excitation light), but the present invention is not limited thereto. In the present invention, the irradiation unit that emits the therapeutic light (excitation light) may be provided as a device separate from the treatment support device. In other words, the treatment support device does not need to be equipped with an irradiation unit that emits therapeutic light (excitation light).

Further, in the above-described embodiment, an example is shown in which the treatment support device 100 is provided with the display unit 90, but the present invention is not limited thereto. In the present invention, the display unit may be provided as a device separate from the treatment support device. In other words, the treatment support device does not need to have a display unit.

### Aspects

It would be understood by those skilled in the art that the exemplary embodiments described above are specific examples of the following aspects.

### (Item 1)

A treatment support device comprising:
a fluorescence detection unit configured to detect, in a photoimmunotherapy for killing cancer cells by irradiating a drug containing a fluorescent substance that has been administered to a body of a subject with therapeutic light in a specific wavelength band, fluorescence emitted from the fluorescent substance of the drug excited by the therapeutic light;
a change information generation unit configured to selectively acquire first signal information corresponding to light in a first wavelength band that is a wavelength band including a wavelength of 770 nm or in the vicinity thereof, among signal waveforms of fluorescence emitted from the fluorescent substance of the drug detected by the fluorescence detection unit, and generate first fluorescence change information that is information on a change in fluorescence intensity in the first wavelength band, based on acquired first signal information; and a notification unit configured to notify progress of a treatment based on irradiation of the therapeutic light, based on the first fluorescence change information.

### (Item 2)

The treatment support device as recited in the above-described Item 1,
wherein the therapeutic light is light in a wavelength band including a wavelength of 690 nm,
wherein the signal waveform of the fluorescence emitted from the fluorescent substance of the drug excited by the therapeutic light has a plurality of peaks, and
wherein the first wavelength band is a wavelength band that includes a first peak positioned at 770 nm or in the vicinity thereof among a plurality of peaks in the signal waveform of the fluorescence emitted from the fluorescent substance of the drug.

### (Item 3)

The treatment support device as recited in the above-described Item 2,
wherein the change information generation unit is configured to generate the first fluorescence change information based on a change in the signal waveform due to an increase in an irradiation time of the therapeutic light in the first wavelength band of the signal waveform of the fluorescence emitted from the fluorescent substance of the drug.

### (Item 4)

The treatment support device as recited in the above-described Item 2 or 3,
wherein the fluorescence detection unit is configured to detect the signal waveform of the light in the first wavelength band from the fluorescence emitted from the fluorescent substance of the drug by sequentially scanning the fluorescence emitted from the fluorescent substance of the drug for each predetermined wavelength band, and
wherein the change information generation unit is configured to acquire the signal waveform of the light in the first wavelength band detected by the fluorescence detection unit as the first signal information by sequentially scanning each predetermined wavelength band and to generate the first fluorescence change information based on the acquired signal waveform of the light in the first wavelength band.

### (Item 5)

The treatment support device as recited in any one of the above-described Items 1 to 4,
wherein the change information generation unit is configured to
selectively acquire, in addition to the first signal information corresponding to the light in the first wavelength band, second signal information corresponding to light in a second wavelength band that is a wavelength band shorter than the first wavelength band and is a wavelength band including a wavelength of 700 nm or more and 730 nm or less, among the signal waveforms of the fluorescence emitted from the fluorescent substance of the drug,
generate second fluorescence change information that is information on a change in fluorescence intensity in the second wavelength band, based on the acquired second signal information, and
generate a treatment progress index that is an index of progress of the treatment based on the first fluorescence change information and the second fluorescence change information.

### (Item 6)

The treatment support device as recited in the above-described Item 5,
wherein the second wavelength band is a wavelength band that includes a rising portion of a second peak positioned in a wavelength band shorter than 770 nm in wavelength, among a plurality of peaks in a signal waveform of the fluorescence emitted from the fluorescent substance of the drug.

### (Item 7)

The treatment support device as recited in the above-described Item 5 or 6,
wherein the change information generation unit is configured to generate the second fluorescence change information, based on the change in the signal waveform due to an increase in the irradiation time of the therapeutic light in the second wavelength band of the signal waveform of the fluorescence emitted from the fluorescent substance of the drug.

### (Item 8)

The treatment support device as recited in any one of the above-described Items 5 to 7,
wherein the change information generation unit is configured to calculate the treatment progress index based on a ratio of the first fluorescence change information to the second fluorescence change information.

### (Item 9)

The treatment support device as recited in any one of the above-described Items 5 to 8,
wherein the fluorescence detection unit is configured to detect each of a signal waveform of light in the first wavelength band and a signal waveform of light in the second wavelength band from the fluorescence emitted from the fluorescent substance by sequentially scanning the fluorescence emitted from the fluorescent substance of the drug for each predetermined wavelength band, and
wherein the change information generation unit is configured to
acquire the signal waveform of light in the second wavelength band detected by the fluorescence detection unit as the second signal information by sequentially scanning the fluorescence for each predetermined wavelength band, and
generate the second fluorescence change information based on the acquired signal waveform of the light in the second wavelength band.

### (Item 10)

The treatment support device as recited in the above-described Item 9, further comprising:
a fluorescence imaging unit provided separately from the fluorescence detection unit, the fluorescence imaging unit being configured to image a distribution of the fluorescence emitted from the fluorescent substance of the drug excited by the therapeutic light, based on light in a wavelength band that includes the first wavelength band and the second wavelength band,
wherein the notification unit includes a display unit that displays at least one of the treatment progress index which is an index of the progress of the treatment based on the first fluorescence change information and the second fluorescence change information and a fluorescence distribution image which is an image showing the distribution of the fluorescence imaged by the fluorescence imaging unit.

### DESCRIPTION OF REFERENCE SYMBOLS

20, 220: Fluorescence detection unit
31: Fluorescence imaging unit
70: PC (Change Information Generation Unit)
90: Display unit (Notification Unit)
91: Fluorescence Distribution Image
91a: Fluorescence Distribution
100, 200, 300, 400: Treatment Support Device
800: Patient (Subject)
801: Cancer cell
900: Drug
901: Fluorescent substance
B1: First wavelength band
B2: Second wavelength band
C1: First fluorescence change information
C2: Second fluorescence change information
C3: Treatment progress index
P1: Peak (first peak)
P2: Peak (second peak)
t1 to t6: Treatment time

1. A treatment support device comprising:
a fluorescence detection unit configured to detect, in a photoimmunotherapy for killing cancer cells by irradiating a drug containing a fluorescent substance that has been administered to a body of a subject with therapeutic light in a specific wavelength band, fluorescence emitted from the fluorescent substance of the drug excited by the therapeutic light;
a change information generation unit configured to selectively acquire first signal information corresponding to light in a first wavelength band that is a wavelength band including a wavelength of 770 nm or in the vicinity thereof, among signal waveforms of fluorescence emitted from the fluorescent substance of the drug detected by the fluorescence detection unit, and generate first fluorescence change information that is information on a change in fluorescence intensity in the first wavelength band, based on acquired first signal information; and
a notification unit configured to notify progress of a treatment based on irradiation of the therapeutic light, based on the first fluorescence change information.

2. The treatment support device as recited in claim 1,
wherein the therapeutic light is light in a wavelength band including a wavelength of 690 nm,
wherein the signal waveform of the fluorescence emitted from the fluorescent substance of the drug excited by the therapeutic light has a plurality of peaks, and
wherein the first wavelength band is a wavelength band that includes a first peak positioned at 770 nm or in the vicinity thereof among a plurality of peaks in the signal waveform of the fluorescence emitted from the fluorescent substance of the drug.

3. The treatment support device as recited in claim 2,
wherein the change information generation unit is configured to generate the first fluorescence change information based on a change in the signal waveform due to an increase in an irradiation time of the therapeutic light in the first wavelength band of the signal waveform of the fluorescence emitted from the fluorescent substance of the drug.

4. The treatment support device as recited in claim 1,
wherein the fluorescence detection unit is configured to detect the signal waveform of the light in the first wavelength band from the fluorescence emitted from the fluorescent substance of the drug by sequentially scanning the fluorescence emitted from the fluorescent substance of the drug for each predetermined wavelength band, and
wherein the change information generation unit is configured to acquire the signal waveform of the light in the first wavelength band detected by the fluorescence detection unit as the first signal information by sequentially scanning each predetermined wavelength band and to generate the first fluorescence change information based on the acquired signal waveform of the light in the first wavelength band.

5. The treatment support device as recited in claim 1,
wherein the change information generation unit is configured to
selectively acquire, in addition to the first signal information corresponding to the light in the first wavelength band, second signal information corresponding to light in a second wavelength band that is a wavelength band shorter than the first wavelength band and is a wavelength band including a wavelength of 700 nm or more and 730 nm or less, among the signal waveforms of the fluorescence emitted from the fluorescent substance of the drug,
generate second fluorescence change information that is information on a change in fluorescence intensity in the second wavelength band, based on the acquired second signal information, and
generate a treatment progress index that is an index of progress of the treatment based on the first fluorescence change information and the second fluorescence change information.

6. The treatment support device as recited in claim 5,
wherein the second wavelength band is a wavelength band that includes a rising portion of a second peak positioned in a wavelength band shorter than 770 nm in wavelength, among a plurality of peaks in a signal waveform of the fluorescence emitted from the fluorescent substance of the drug.

7. The treatment support device as recited in claim 5,
wherein the change information generation unit is configured to generate the second fluorescence change information, based on the change in the signal waveform due to an increase in the irradiation time of the therapeutic light in the second wavelength band of the signal waveform of the fluorescence emitted from the fluorescent substance of the drug.

8. The treatment support device as recited in claim 5,
wherein the change information generation unit is configured to calculate the treatment progress index based on a ratio of the first fluorescence change information to the second fluorescence change information.

9. The treatment support device as recited in claim 5,
wherein the fluorescence detection unit is configured to detect each of a signal waveform of light in the first wavelength band and a signal waveform of light in the second wavelength band from the fluorescence emitted from the fluorescent substance by sequentially scanning the fluorescence emitted from the fluorescent substance of the drug for each predetermined wavelength band, and
wherein the change information generation unit is configured to
acquire the signal waveform of light in the second wavelength band detected by the fluorescence detection unit as the second signal information by sequentially scanning the fluorescence for each predetermined wavelength band, and
generate the second fluorescence change information based on the acquired signal waveform of the light in the second wavelength band.

10. The treatment support device as recited in claim 9, further comprising:
a fluorescence imaging unit provided separately from the fluorescence detection unit, the fluorescence imaging unit being configured to image a distribution of the fluorescence emitted from the fluorescent substance of the drug excited by the therapeutic light, based on light in a wavelength band that includes the first wavelength band and the second wavelength band,
wherein the notification unit includes a display unit that displays at least one of the treatment progress index which is an index of the progress of the treatment based on the first fluorescence change information and the second fluorescence change information and a fluorescence distribution image which is an image showing the distribution of the fluorescence imaged by the fluorescence imaging unit.
